(19) United States
(12) Patent Application Publication
(10) Pub. No.: US 2005/0064509 A1
(43) Pub. Date: Mar. 24, 2005

(54) Use of templated self assembly to create novel multifunctional species

(75) Inventors: Andrew Bradbury, Santa Fe, NM (US); Geoff Waldo, Santa Fe, NM (US); Peter Pavlik, Los Alamos, NM (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/670,167

(22) Filed: Sep. 23, 2003

(51) Int. Cl. G01N 33/53; G06F 19/00; C07K 14/435

(52) U.S. Cl. 435/7.1; 530/350

(57) ABSTRACT

The present invention provides self-assembling bifunctional polypeptides, kits comprising the self assembling bifunctional polypeptides, methods for assembling the bifunctional polypeptides, and methods for screening for the presence of an antigen using the bifunctional polypeptides.

### STATEMENT AS TO RIGHTS TO INVENTIONS MADE UNDER FEDERALLY SPONSORED RESEARCH AND DEVELOPMENT

This invention was made with government support under grant number DE-FG02-98ER62647 from the United States Department of Energy and Contract No. W-7405-ENG-36 awarded by the United States Department of Energy to The Regents of The University of California. The government has certain rights in this invention.

### BACKGROUND OF THE INVENTION

Bifunctional and multi-functional polypeptides which combine the functions of one or more polypeptides (i.e., binding functions and reporter functions) are useful for many applications. For example, polypeptides with binding functions such as antibodies may be linked to reporter proteins (e.g. fluorescent proteins, luminescent proteins, colored proteins, and enzymes) or reporter dyes (e.g., fluorescent dyes, radiolabels) for in vitro and in vivo use in detecting the presence of a particular antigen in a sample (see, e.g., Pluckthun and Pack, Immunotechnology 3:830105 (1997); Rheinnecker et al., J. Immunol. 157:2989-2997 (1996); Lindner et al., Biotechniques 22:140-149 (1997); Ducancel et al., Biotechnology 11:601-605 (1992); Wels et al., Biotechnology 10:1128-1132 (1992)). Antibodies linked to therapeutic agents (e.g., radioisotopes, chemotherapeutic drugs, ribozymes, and toxins) may also be used to deliver or selectively localize the agents to particular cells, organs, or tissues (see, e.g., Pluckthun and Pack, 1997, supra; Rheinnecker et al., 1996, supra; Sung and van Odsel, J. Nucl. Med. 36:867-876 (1995)).

Current methods of generating bifunctional fusion proteins typically use recombinant DNA technology or chemical conjugation. Each method has drawbacks. For example, creation of bifunctional fusion proteins using recombinant DNA methodology often leads to reduced protein expression and decreased protein folding efficiency. One exemplary bifunctional protein is scFv-GFP. The scFv is synthesized most efficiently in the periplasmic space while GFP is most efficiently synthesized in the cytoplasm. Thus, if the fusion scFv-GFP protein is expressed in a single cell, the expression or folding of either the scFv component or the GFP component will be compromised. Likewise, chemical conjugation of two polypeptides to create a single bifunctional polypeptide is a complicated procedure: it is difficult to control the site of attachment of the functional group and to control the number of functional groups attached. Bifunctional and multifunctional polypeptides which can be assembled without these complications are therefore needed. This invention addresses that need.

### SUMMARY OF THE INVENTION

The present invention provides self-assembling bifunctional and multifunctional polypeptides, kits comprising the polypeptides, methods for assembling the polypeptides, and methods for screening for the presence of an antigen or target molecule using the polypeptides.

In one embodiment, the present invention provides a bifunctional polypeptide comprising a binding ligand linked to a first member of a coil-coil binding pair and a reporter molecule linked to the second member of a coil-coil binding pair, wherein binding between the first coil domain and the second coil domain joins the binding ligand to the reporter molecule.

In another embodiment, the present invention provides a kit comprising: a binding ligand linked to a first member of a coil-coil binding pair; and a reporter molecule linked to a second member of a coil-coil binding pair.

In another embodiment, the present invention provides a method of assembling a bifunctional polypeptide. A binding ligand linked to a first member of a coil-coil binding pair and a reporter molecule linked to a second member of a coil-coil binding pair are incubated under conditions in which the first binding pair member specifically binds to the second binding pair member, thereby assembling the bifunctional polypeptide.

In an additional embodiment, the present invention provides a method of screening for the presence of an antigen or target molecule that binds to the binding ligand. A sample comprising the antigen is incubated with a bifunctional polypeptide comprising a binding ligand linked to a first member of a coil-coil binding pair and a reporter molecule linked to the second member of the coil-coil binding pair. The binding ligand is joined to the reporter by the binding interaction between the binding pair members. The antigen and the bifunctional polypeptide are incubated under conditions in which the antigen specifically binds to the binding ligand. Reporter activity is detected, thereby detecting the presence of the antigen.

In a further embodiment, the present invention provides a method of screening for the presence of an antigen (or target molecule that binds to a binding ligand). A sample comprising the antigen is incubated with binding ligand linked to a first member of a coil-coil binding pair under conditions in which the antigen specifically binds to the binding ligand. The sample is subsequently incubated with a reporter molecule linked to the second member of the coil-coil binding pair. The binding ligand becomes joined to the reporter molecule by the binding interaction between the binding pair members. Activity of the reporter molecule is detected, thereby detecting the presence of the antigen.

In another aspect, the invention provides a bifunctional polypeptide comprising one polypeptide linked to a first member of a coil-coil binding pair and a second polypeptide linked to the second member of a coil-coil binding pair, wherein binding between the first coil domain and the second coil domain joins the two polypeptides, and wherein the first polypeptide is a binding ligand, and the second polypeptide is a polypeptide which undergoes spontaneous multimerization, wherein such multimerization involves the spontaneous association of n units, wherein n is 2 or more. The polypeptide that undergoes multimerization may be, e.g., ferritin, multi-enzyme complexes (such as the E2 polypeptide from the pyruvate dehydrogenase multienzyme complex of Bacillus stearothermophilus), viral coat proteins derived from viruses such as poliovirus, Hepatitis B, Cow pea mosaic virus, Johnson Grass Mosaic Virus, polyoma viruses of many species, and nodaviruses of different species, or another spontaneously assembling polypeptide sequence. In some cases, self assembly requires a single polypeptide, while in other cases, more than one polypeptide is required.

In another aspect, the invention provides a number of bifunctional polypeptides, each comprising one polypeptide linked to a first member of a coil-coil binding pair and a second polypeptide linked to the second member of a coil-coil binding pair, wherein binding between the first coil domain and the second coil domain joins the two polypeptides, and wherein the first polypeptide is a binding ligand or a reporter protein, and the second polypeptide is a polypeptide which undergoes spontaneous multimerization, wherein such multimerization involves the spontaneous association of n units, wherein n is 2 or more. The polypeptide that undergoes multimerization may be, e.g., ferritin, a viral coat protein, or another spontaneously assembling polypeptide sequence. Such an aspect provides for linkage between binding activity and reporter activity by a multimerizing protein, wherein either binding activity or reporter activity may be more or less represented, the former providing for greater avidity, and the latter for greater reporter activity.

Thus, in some embodiments, the invention provides a multifunctional polypeptide comprising: a first member of a coil-coil binding pair individually linked to one or more binding ligands; and a second member of the coil-coil binding pair linked individually linked to one or more polypeptides that undergoes spontaneous multimerization, to form a self-assembled complex; wherein binding between the first member of the coil-coil binding pair and the second member of the coil-coil pair join the binding ligand, or binding ligands, and the self-assembled complex. The multifunctional polypeptide can further comprise a subunit that is a reporter molecule linked to a first member of the coil-coil binding pair, wherein the coil-coil binding interactions join the self-assembled complex to the binding ligand, or binding ligands, and the reporter molecule. The spontaneously mutimerizing polypeptide can be, e.g., a soluble ferritin, or a viral coat protein

In some embodiments, the reporter molecules that are contained in the bifunctional or multifunctional polypeptides of the invention are polypeptides, e.g., fluorescent proteins, such as green or red fluorescent proteins; enzymes, such as horseradish peroxidase, alkaline phosphatase, or β-galactosidase; a biotin binding protein; or an enzyme that has luminescent activity when incubated with an appropriate substrate, e.g., luciferase. In other embodiments, the reporter molecule is a detectable label such as a fluorescent dye or radioactive label.

In some embodiments, the binding ligand is an antibody, e.g, an scFv or an Fab fragment. In other embodiments, the binding ligand is a fluorobody, a chromobody, or a peptide, or a receptor.

The domains of member of a coil-coil binding pair can be a variety of lengths. Typically, each domain is at least 35 amino acids in length.

### BRIEF DESCRIPTION OF THE DRAWINGS

FIG. 1 shows different bifunctional and multi-functional species prepared according to the methods of the invention. Coiled coils are used to link binding ligands, reporter molecules and multimerization domains in different combinations.

FIG. 2 depicts labeling single chain Fvs with fluorescent organic dyes using coil peptides. The scFv with an E coil at its C terminus binds to a K coil labeled with a fluorescent dye. The shift in mobility of the intense dye band shows that this labeling has occurred. When antigen is added, the band shifts further, indicating that the antigen has been recognized by the scFv labeled with the dye by coils.

FIG. 3 shows labeling scFvs with GFP using coil-GFP peptide fusions. The scFv is labeled with GFP using coiled coils. The addition of the scFv to the western blot, followed by scanning (lanes 5-8) allows the detection of the same bands as those recognized using typical sandwhich detection with secondary labeled antibodies (lanes 1-4).

FIG. 4 shows fluorescent resonant energy transfer using scFvs labeled with GFP and BFP using coil fusions. Two E coil—scFv fusions of two scFvs (D1.3 and HyHEL10) recognizing lysozyme were created. These were purified and mixed with K coil GFP or K coil BFP respectively. The purified fluorescent protein labeled scFvs were mixed with different amounts of the recognized antigen (lysozyme). The acceptor/donor ratio increases with increasing amounts of antigen, with a sensitivity (in this non-optimized system) of 80 ng lysozyme.

FIG. 5 shows labeling scFvs with alkaline phosphatase using coil-alkaline phosphatase fusions. Alkaline phosphatase is fused to a K or E coil, and used in an enzyme linked immunosorbant assay under the following conditions: 1) Lysozyme coated on well, Kcoil-AP added; 2) Ubiquitin coated on a well, Kcoil-AP added; 3) Lysozyme coated on well, aU4-Ecoil and Ecoil-AP added; 4) Ubiquitin coated on well, aU4-Ecoil and Ecoil-AP added; 5) Lysozyme coated on well, aU4-Ecoil and Kcoil-AP added; 6) Ubiquitin coated on well, aU4-Ecoil and Kcoil-AP added. Only in the case where the scFv recognizes the antigen (aU4 and ubiquitin) and the scFv and AP are labeled with appropriately interacting coils (Ecoil and Kcoil) is a significant signal seen. Abbreviations: AP: alkaline phosphatase; aU4: anti-ubiquitin scFv.

### DETAILED DESCRIPTION OF THE INVENTION

I. Introduction

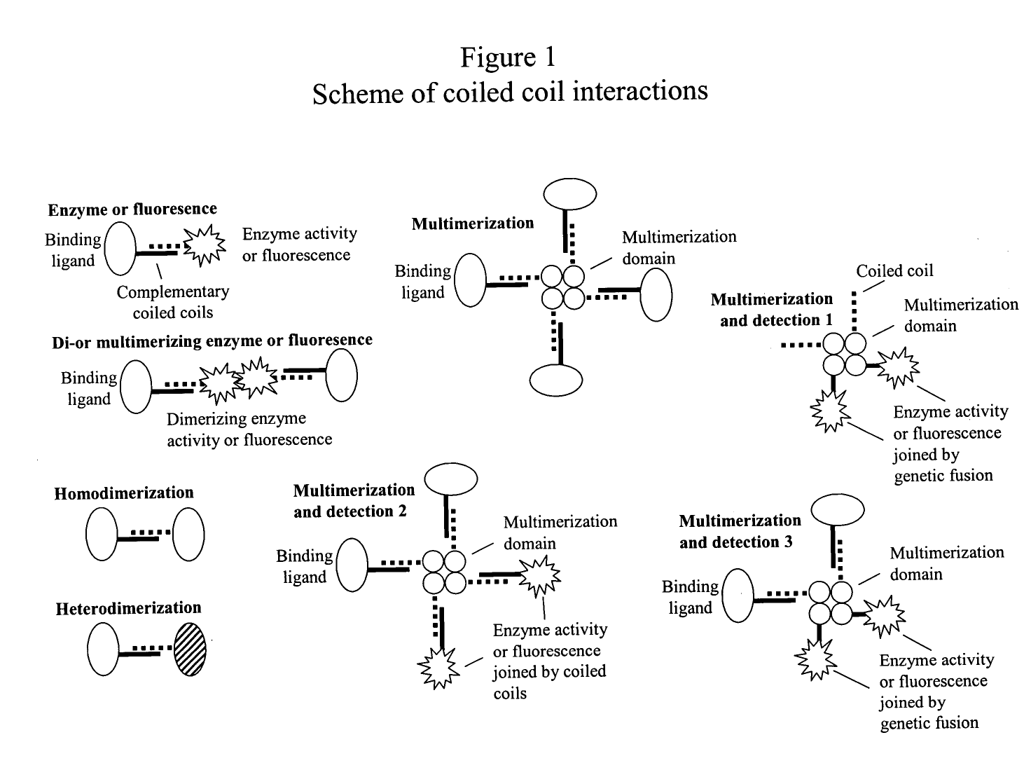

The invention provides self-assembling bifunctional or multifunctional polypeptides (FIG. 1) and kits comprising the polypeptide or subunits of the polypeptides. The invention also methods of screening for the presence of an antigen or a binding target using the bifunctional or multifunctional polypeptides. The polypeptides comprise at least two separate functional domains (e.g., a binding ligand and a reporter molecule) linked by a coiled coil binding interaction.

II. Definitions

“Bifunctional” as used herein refers to a polypeptide that comprises a binding ligand and a molecule, typically a polypeptide, with an activity other than binding, e.g., a reporter molecule, or a spontaneously multimerizing polypeptide, linked by a coiled-coil.

“Multifunctional” refers to polypeptides having multiple domains that are linked to one another by coiled-coil binding interactions. As used herein, “multifunctional” typically refers to a polypeptide that comprises at least one binding ligand, and at least one spontaneously multimerizing polypeptide. In the context of this invention, the term “multifunctional” does not exclude “bifunctional” polypeptides defined above.

“Binding ligand” refers to a polypeptide that specifically binds to a binding target, for example, another polypeptide (such as an antigenic epitope), a nucleic acid, or a lipid. A typical binding ligand is an antibody, a fluorobody, a chromobody, a peptide or a receptor ligand.

A “fluorobody” refers to a binding ligand with intrinsic fluorescence. Exemplary fluorobodies and methods of making them are described in e.g., U.S. patent application Ser. No. 10/132,067, filed Apr. 24, 2002 and Ser. No. 10/423,463, filed Apr. 24, 2003.

A “chromobody” refers to a binding ligand with intrinsic color. Exemplary chromobodies and methods of making them are described, e.g., in U.S. patent application Ser. No. 10/423,463, filed Apr. 24, 2003.

“Antibody” refers to a polypeptide encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition, i.e., the antibody variable region. The terms variable light chain (V_{L}) and variable heavy chain (V_{H}) refer to these light and heavy chains respectively. The antibody variable region comprises three antibody hypervariable regions (also known as complementarity determining regions (CDR's)) and four antibody “framework regions” which flank the CDR's and are conserved. (See, Fundamental Immunology (Paul ed., 4th ed. 1999).

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see, Fundamental Immunology (Paul ed., 4th ed. 1999). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

“Reporter” molecule as used herein refers to any molecule that allows detection of the target of the binding ligand portion of the bifunctional polypeptide described herein. Typical reporter molecules include, for example, fluorescent proteins (e.g., GFP, BFP, dsRed), fluorescent dyes (e.g., rhodamine and its derivatives, dansyl, umbelliferone, fluorescein and its derivatives), luminescent proteins (e.g., luciferase), enzymes (e.g., hydrolases, particularly phosphatases, more particularly alkaline phosphatase, esterases and glycosidases, or oxidases, particularly peroxidases, such as horseradish peroxidase,), biotin binding proteins (e.g., streptavidin and avidin), and radiolabels (e.g., ¹²⁵I, ³²P, ³⁵S, and ³H). For a review of various labels or signal producers that may be used, see U.S. Pat. No. 4,391,904.

A “binding target” or “analyte” in the context of this invention refers to a molecule that specifically binds to a binding ligand.

A “label” or a “detectable moiety” is a composition detectable by spectroscopic, photochemical, biochemical, immunochemical, or chemical means.

“Fluorescent” or “fluorescence ” as used herein refers to luminescence that is caused by the absorption of radiation at one wavelength followed by nearly immediate reradiation usually at a different wavelength and that ceases almost at once when the incident radiation stops. Exemplary fluorescent polypeptides include green fluorescent protein (GFP). The term “green fluorescent protein” as used herein includes variants, such as cyan fluorescent protein, blue fluorescent protein, yellow fluorescent proteins, etc (see, e.g., Ormo et al. Science Sep. 6, 1996;273(5280):1392-5; Yang et al, Nat Biotechnol. October 1996;14(10):1246-51; and U.S. patent application Ser. No. 10/132,067, filed Apr. 24, 2002 and Ser. No. 10/423,463, filed Apr. 24, 2003). Other fluorescent proteins, such as the red fluorescent protein dsRED and variants (Matz et al., Nat. Biotechnol. 17:969-973, 1999; U.S. patent application Ser. Nos. 10/132,067 and 10/423,463, supra), can also be used.

A “chromophoric protein” refers to a protein that has intrinsic color. Examples of chromophoric proteins are provided in U.S. patent application Ser. No. 10/423,463, filed Apr. 24, 2003.

“Luminescent” or “luminescence” as used herein refers to the low-temperature emission of light by a chemical or physiological process, i.e., chemiluminescence or bioluminescence. Exemplary luminescent polypeptides include luciferase.

“Binding pair” refers to a pair of coils that self assemble to form a coiled-coil.

“Coil-coil” or “coiled coil” as used herein refers to an α-helical oligomerization domain found in a variety of proteins. Proteins with heterologous domains joined by coiled coils are described in U.S. Pat. Nos. 5,716,805 and 5,837,816. Structural features of coiled-coils are described in Litowski and Hodges, J. Biol. Chem. 277:37272-27279, 2002; Lupas TIBS 21:375-382 (1996); Kohn and Hodges TIBTECH 16: 379-389(1998); and Müller et al. Methods Enzymol. 328: 261-282 (2000). Coiled-coils generally comprise two to five α-helices (see, e.g., Litowski and Hodges, 2002, supra). The α-helices may be the same or difference and may be parallel or anti-parallel. Typically, coiled-coils comprise an amino acid heptad repeat: “abcdefg.” “Coiled-coil” domains are described in greater detail below.

The phrase “specifically (or selectively) binds” when used in reference to binding between coiled-coil binding pair members, e.g., an E coil and a K coil or an A coil and a B coil, refers to the coil-coil interaction that assembles the bifunctional or multifunctional polypeptide. Thus, under designated incubation conditions for self assembly, the specified coiled coil binding pair members bind to their specific binding partner at least two times the background (i.e., nonspecific binding to polypeptides) and more typically more than 10 to 100 times background. For example a K coil and an E coil bind at a K_{d}=6×10⁻¹¹ to 1×10⁻⁹(M) (Crescenzo, supra) and an A coil and a B coil bind at a K_{d} of 2.4×10⁻⁸ (M) (Arndt, supra). Temperatures can range from 0° C. to 60° C.; moderate to low salt concentration <500 mM NaCl, pH between 5 and 10 (see Crescenzo, supra and Arndt, supra, 2003).

The term “spontaneous multimerization” in the context of this invention refers to the ability of a polypeptide to spontaneously adopt a quaternary structure and is applied to molecules that spontaneously assemble into a complex of at least two molecules. Such molecules may be fused to coiled coils permitting the effective multimerization of those proteins fused to the coiled coil pairs.

The phrase “specifically (or selectively) binds” when used in reference to an antibody, fluorobody, or chromobody; or “specifically (or selectively) immunoreactive with,” when referring to a protein or peptide, refers to a binding reaction that is determinative of the presence of the protein, often in a heterogeneous population of proteins and other biologics. Specific binding to an antibody, fluorobody, or chromobody under such conditions requires an antibody, fluorobody, or chromobody that is selected for its specificity for a particular protein. For example, polyclonal antibodies raised to a particular protein, polymorphic variants, alleles, orthologs, and conservatively modified variants, or splice variants, or portions of the particular protein, can be selected to obtain only those polyclonal antibodies that are specifically immunoreactive with the particular protein and not with other proteins. This selection may be achieved by subtracting out antibodies that cross-react with other molecules. A similar approach may be employed to select specifically immunoreactive fluorobodies or chromobodies. A variety of immunoassay formats may be used to select antibodies, flourobodies, or chromobodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

“Assembling” or “assembly” as used herein in the context of joining coil-coil binding pairs refers to combining polypeptides comprising at least one binding ligand linked to a first member of a coiled-coil binding pair and at least one other molecule, e.g., a reporter molecule, linked to the second member of a coiled-coil binding pair under conditions sufficient to allow attachment of the polypeptides via their coils. For example, a scFv linked to an E coil is mixed with GFP linked to a K coil under conditions in which the E coil and the K coil interact and assemble to form a bifunctional scFV-GFP polypeptide, for example, mixing in Dulbecco's Phosphate Buffered Saline (PBS) at room temperature for 15 minutes.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean all of a first sequence is complementary to at least a portion of a reference polynucleotide sequence.

Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. The percent identity between two sequences can be represented by any integer from 25% to 100%. More preferred embodiments include at least: 25%, 30%, 35%, 40%, 45%, 50%, 55%, 60%, 65%, 70%, 75%, 80%, 85%, 90%, 95%, or 99%.

One example of an algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Mixed nucleotides are designated as described in e.g. Eur. J. Biochem. (1985) 150:1.

“Heterologous”, when used with reference to portions of a protein, indicates that the protein comprises two or more domains that are not found in the same relationship to each other in nature. Such a protein, e.g., a fusion protein or a conjugate protein, contains two or more domains from unrelated proteins arranged to make a new functional protein. Heterologous may also refer to a natural protein when it is found or expressed in an unnatural location such as when a mammalian protein is expressed in a bacterial cell. A heterologous polypeptide may correspond to a single known protein (e.g. GFP) or may itself be a heterologous protein composed of domains or portions of multiple different proteins.

“Homologous”, when used with reference to portions of a protein, indicates that the protein comprises two or more domains that are found in the same relationship to each other in nature (e.g. antibody hypervariable regions and antibody framework regions). A homologous protein may correspond to one or more domain or portion of single known protein arranged in their native order or rearranged.

“Nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

“Codon” refers to a nucleotide sequence that specifies an amino acid or represents a signal to initiate or stop a function. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605 (1985); Rossolini et al., Mol. Cell. Probes 8:91 (1994)). The term nucleic acid is used interchangeably with gene, cDNA, mRNA, oligonucleotide, and polynucleotide.

The term “nucleic acid encoding” or “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both full length nucleic acid sequences as well as shorter sequences derived from the full length sequences. It is understood that a particular nucleic acid sequence includes the degenerate codons of the native sequence or sequences which may be introduced to provide codon preference in a specific host cell. The nucleic acid includes both the sense and antisense strands as either individual single strands or in the duplex form.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

“Promoter” and “expression control sequence” are used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

“Polypeptide,” “peptide” and “protein” are used interchangeably herein to refer to a polymer of amino acid residues. The terms apply to naturally occurring amino acid polymers, as well as, amino acid polymers in which one or more amino acid residue is an artificial chemical mimetic of a corresponding naturally occurring amino acid.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

“Conservatively modified variants” applies to both nucleic acid and amino acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, E or D, may be substituted with its uncharged counterpart, Q or N, respectively; or vice versa. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the covalent or noncovalent association of independent tertiary units.

The terms “isolated” or “substantially purified,” when applied to a nucleic acid or protein, denotes that the nucleic acid or protein is essentially free of other cellular components with which it is associated in nature. An isolated nucleic acid or protein is preferably in a substantially omogeneous state, although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified.

The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure. The term “nucleic acid” refers to a deoxyribonucleotide or ribonucleotide polymer in either single- or double-stranded form, and unless otherwise limited, would encompass known analogs of natural nucleotides that can function in a similar manner as naturally occurring nucleotides.

The term “pharmaceutical composition” refers to formulations of various preparations. Parenteral formulations are known and are preferred for use in the invention. The formulations containing therapeutically effective amounts of multifunctional proteins are either sterile liquid solutions, liquid suspensions or lyophilized versions and optionally contain stabilizers or excipients. Lyophilized compositions are reconstituted with suitable diluents, e.g., water for injection, saline, 0.3% glycine and the like, at about 0.01 mg/kg of host body weight to about 10 mg/kg or more host body weight.

A “therapeutically effective amount” of a polypeptide of the invention is an amount sufficient to provide a therapeutic effect, i.e., an amount of polypeptide effective for delivering the desired amount of a therapeutic agent to a cell, organ, or tissue (e.g., an amount effect for inhibiting growth of malignant cells).

III. Linkage of Polypeptides, Reporters, and Coiled-Coil Binding Pair Members.

One embodiment of the present invention provides bifunctional or multifunctional polypeptides comprising a binding ligand and a reporter molecule joined by a coiled-coil structure. For example, a first subunit can be the binding ligand linked to a first member of a coiled-coil binding pair and a second subunit can be a reporter molecule linked to a second member of a coiled-coil binding pair. Once the linked binding ligand and linked reporter molecule are generated, the two subunit polypeptides will assemble, to form a bifunctional polypeptide as described herein. A subunit comprising a polypeptide capable of self-assembly linked to a member of a coiled-coil binding pair may also constitute part of a bifunctional or multifunctional polypeptide of the invention. It is also joined to one or more additional subunit polypeptides, e.g., a binding ligand-coil subunit polypeptide, through coiled-coil binding.

Coiled-coils generally comprise two to five α-helices (see, e.g., Litowski and Hodges, 2002, supra). The α-helices may be the same or different and may be parallel or anti-parallel. Typically, coiled-coils comprise an amino acid heptad repeat: “abcdefg.” Side chains from amino acids a and d pack against each other to form a continuous hydrophobic core along the length of the α-helices. The side chains of amino acids e and g are along the side of the hydrophobic cored. Amino acids e and g are typically charged residues that participate in electrostatic interactions which specify homo- and hetero-association between coils. The exposed amino acids b, c, e, f, and g affect the α-helical propensities of the coil.

Amino acids a and d are generally hydrophobic residues that form the hydrophobic core of the α-helices, for example, valine, leucine, isoleucine, methionine, tyrosine, tryptophan, or phenylalanine. Serine can also be used to form ‘serine zippers’ (Adamian & Liang, Proteins 47:209-218, 2002). Amino acids e and g are typically charged residues and are occupied by glutamic acid in the E coils and lysine in the K coils.

Exemplary coiled-coils include E coils and K coils associated 1:1 to form a heterodimer, A coils and B coils associated 1:1 to form a heterodimer, and other leucine zippers. Typically, the 5 heptad E and K (i.e., E/K) coiled coil exhibits a stability of ΔG=−14.0 kcal/mol and a dissociation constant of K_{d}=6×10⁻¹¹ to 1×10⁻⁹(M). Shorter E and K coils (a 4 heptad E coil binding to a 3 heptad K coil) exhibit a stability of ΔG=−6 to −8 kcal/mol and a dissociation constant of K_{d}=2.3×10⁻⁵ (M) (De Crescenzo et al., Biochemistry 42:1754-1763, 2003). Typically, the A and B (i.e., A/B) coiled coil exhibit a dissociation constant of 2.4×10⁻⁸ (M) (Arndt et al, J. Mol. Biol. 295:627-639, 2000). E coils and K coils are described in detail in Litowski and Hodges, supra. Preferred E coils generally comprise multimers of the sequence: VSALEKE. Preferred K coils generally comprise multimers of the sequence VSALKEK. The valine residues can be substituted by isoleucine; the alanine residues can be substituted by serine (Litowski and Hodges, supra). Preferred A coils generally comprise the sequence VAQLEEKVKTLRAQNYELKSRVQRLREQVAQL and preferred B coils generally comprise the sequence VDELQAEVDQLQDENYALKTKVAQLRKKVEKL. Typically, the E and K coils or A and B coils are at least 14 amino acids in length, even more typically at least 21 amino acids in length. Often the E and K coils or A and B coils are 35 (E/K) or 32 (A/B) amino acids in length, i.e., about 5 heptad repeats. Generally, 35 amino acids is the length used. The longer the coil the greater the expected affinity.

Those of skill in the art will understand that multiple amino acid substitutions may be made that do not affect the stability or α-helical propensities of the coiled coils. Such mutations may be identified either by mutation and selection experiments, or by rational design methods, both of which are described, by way of example, in Arndt et al., (Structure (Camb) September 2002;10(9):1235). In vivo mutation and selection experiments occasionally identify unexpected residues which improve the function of the coils, and in general have identified better coils than those designed rationally, although the nature of the selection experiment will determine the nature of the coils selected. If there is no counter-selection for homodimerization between the coils, the affinity for such homodimers may also increase during the selective process. Likewise, multiple amino acid substitutions may be made to enhance the stability or α-helical propensities of the coiled coils. For example, the amino acid isoleucine may be substituted into the a position of an E or K coil to increase the hydrophobicity of the coil, and the amino acid alanine may be substituted into the b position of an E or K coil to increased the α-helical propensities of the coiled coils (see, e.g., Litowski and Hodges, 2002).

In some embodiments, the binding ligand is an antibody, including, e.g., scFv, heavy or light chain variable regions, and fragments. The binding ligand may also be a fluorobody, a chromobody, a receptor or a ligand of a receptor. In other embodiments, the invention provides a bifunctional polypeptide that comprises a binding ligand linked to one of the coil domains with the second coil domain linked to a polypeptide that undergoes spontaneous multimerization. In this invention, a multimerized complex comprises at least two, typically, three or more polypeptide subunits. A number of polypeptides have this capability, including, e.g., ferritin and viral coat proteins derived from viruses such as poliovirus, Hepatitis B, Cow pea mosaic virus, Johnson Grass Mosaic Virus, polyoma viruses of many species, and nodaviruses of different species. In some cases, self assembly requires a single polypeptide, while in other cases, more than one polypeptide is required.

In the case of a multimerized fluorobody or chromobody mediated by coil-coil interactions, the signaling element and the binding element are the same element.

The bifunctional or multifunctional polypeptides and their components can be generated by any means known in the art. For example, the linkage between the binding ligand and the member of the coiled-coil binding pair (e.g. the binding ligand and an E coil) and the linkage between the reporter peptide and the member of the coiled-coil binding pair (e.g., the reporter peptide and a K coil) may be introduced through recombinant means or chemical means.

A. Recombinant Linkages

Recombinant methods of introducing linkages between polypeptides are well known to those of skill in the art. For example, routine techniques in the field of recombinant genetics may be used to introduce the linkages. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

An amino acid linker sequence may be employed to separate the binding ligand, multimeric domain, or reporter molecule from their respective coils by a distance sufficient to ensure that each polypeptide folds correctly into its secondary and tertiary structures. Such an amino acid linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Typical peptide linker sequences contain Gly, Val and Thr residues. Other near neutral amino acids, such as Ser and Ala can also be used in the linker sequence. Amino acid sequences which may be usefully employed as linkers include those disclosed in Maratea et al. (1985) Gene 40:39-46; Murphy et al. (1986) Proc. Natl. Acad. Sci. USA 83:8258-8262; U.S. Pat. Nos. 4,935,233 and 4,751,180. The linker sequence may generally be from 1 to about 50 amino acids in length, e.g., 3, 4, 6, or 10 amino acids in length, but can be 100 or 200 amino acids in length. Linker sequences may not be required when the first and second polypeptides have non-essential N-terminal amino acid regions that can be used to separate the functional domains and prevent steric interference.

For example, in specific embodiments, further described in the Examples, infra, the scFv-coil fusions were constructed both in pET27b (Novagen; Kanamycin resistance, pBR322 origin) and in pDAN5 (Sblattero & Bradbury, Nature Biotech. 18:75-80, 2000; Ampicillin resistance, pUC origin). For pET27b constructs, scFv's selected from a phagemid antibody library (Sheets, et al., Proc. Natl. Acad. Sci. USA 95:6157-6162, 1998) were subloned into pET27b using NcoI and NotI restriction sites, then coil sequences with linkers at both ends were cloned in using XhoI and NheI restriction sites. Exemplary amino acid linkers separating the scFv and coil are as follows: scFv sequence, Ala, Ala, Ala (NotI), Leu, Glu (XhoI), Gly, Gly, Gly, Ser, Gly, Gly, Gly, Ser, coil sequence, Gly, Gly, Gly, Ser, Gly, Gly, Gly, Ser, Ala, Ser (NheI), with restriction sites in brackets. For pDAN5 constructs, coil sequences with linkers at both sides were cloned in using the NheI restriction site.

The amino acid linkers separating the scFv and coil in these exemplary constructus are as follows: scFv sequence, Ala, Ser (NheI), Ser, Gly, Gly, Gly, Gly, Ser, Glu, Asn, Ala, Ser, Pro, coil sequence, Gly, Gly, Gly, Ser, Glu, Ser, Gly, Thr, Ser (SpeI/NheI).

In another specific embodiment, also further described in the Examples, infra, an N-terminal alkaline phosphatase (AP) coil fusion was constructed in pSKAP/S vector (Griep, et al., Prot. Expr. Pur. 16:63-69, 1999). The vector has a ColE1 origin of replication, ampicillin resistance and the AP fusion gene is under the control of the TetA promoter. Coil sequences with linker at both sides were cloned in pSKAP/S using SfiI and NotI restriction sites. The amino acids surrounding the coils are as follows: Ala, Ala, Gln, Pro, Ala (SfiI), Leu, Ala, Gly, Gly, Ser, Glu, Asn, Ala, Ser, Pro, coil sequence, Gly, Gly, Gly, Ser, Glu, Ser, Gly, Ala, Ala, Ala (NotI), AP sequence.

1. Expression in Prokaryotes and Eukaryotes

To obtain high level expression of a cloned gene, such as those cDNAs encoding, for example, a coil domain or a binding ligand, a reporter polypeptide, or a self-multimerizing domain, either individually or joined to a coil such as a K coil or an E coil. One typically subclones the desired cDNA into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Bacterial expression systems for expressing the protein of interest are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence and signals required for efficient polyadenylation of the transcript, ribosome binding sites, and translation termination. The termination region may be obtained from the same gene as the promoter sequence or may be obtained from different genes. The nucleic acid sequence may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

The particular expression vector used to transport the genetic information into the cell is not critical. Any conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a nucleic acid sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein or polypeptide, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the polypeptide of interest.

After the expression vector is introduced into the cells, the transfected cells are cultured under conditions favoring expression of the protein of interest which is recovered from the culture using standard techniques identified below.

The recombinant protein or polypeptide is purified from any suitable expression system by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant proteins are being purified. For example, proteins having established molecular adhesion properties can be reversible fused to the protein of interest. With the appropriate ligand, the protein of interest can be selectively adsorbed to a purification column and then freed from the column in a relatively pure form. The fused protein is then removed by enzymatic activity. Finally the protein of interest could be purified using immunoaffinity columns.

Recombinant proteins are expressed by transformed bacteria in large amounts, typically after promoter induction; but expression can be constitutive. Promoter induction with IPTG is a one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of protein from inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. The protein of interest is separated from other bacterial proteins by standard separation techniques, e.g., with Ni—NTA agarose resin.

Alternatively, it is possible to purify protein from bacteria periplasm. After lysis of the bacteria, when protein is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Chemical Linkage

Chemical linkages known in the art may be used to join or link a domain, e.g., a binding ligand, self-assembling polypeptide, or reporter molecule, to a member of the coiled-coil binding pair. Exemplary chemical linkages include, for example, covalent bonding, including disulfide bonding; hydrogen bonding; electrostatic bonding; recombinant fusion; and conformational bonding, e.g., antibody-antigen, biotin-avidin associations, digoxigenin-anti-digoxigenin and associations. Additional linkers and methods of linking are described in WO 98/41641 and U.S. Pat. No. 5,852,178.

Chemical means of joining the binding ligand or reporter polypeptide to their respective coils are described, e.g., in Bioconjugate Techniques, Hermanson, Ed., Academic Press (1996). Chemical modifications include, for example, derivitization for the purpose of linking the binding ligand and the first coil or the reporter polypeptide and the second coil to each other, either directly or through a linking compound, by methods that are well known in the art of protein chemistry. For example, a heterobifunctional coupling reagent which ultimately contributes to formation of an intermolecular disulfide bond between the binding ligand or the reporter peptide and their respective coils. Other types of coupling reagents that are useful in this capacity for the present invention are described, for example, in U.S. Pat. No. 4,545,985.

The means of linking the binding ligand or a reporter polypeptide and their respective coils may also use thioether linkages between heterobifunctional crosslinking reagents or specific low pH cleavable crosslinkers or specific protease cleavable linkers or other cleavable or noncleavable chemical linkages. The means of linking the binding ligand or the reporter peptide and their respective coils may also comprise a peptidyl bond formed between the binding ligand or the reporter peptide and their respective coils synthesized by standard peptide synthesis chemistry. The protein itself can also be produced using chemical methods to synthesize an amino acid sequence in whole or in part. For example, peptides can be synthesized by solid phase techniques, such as, e.g., the Merrifield solid phase synthesis method, in which amino acids are sequentially added to a growing chain of amino acids (see, Merrifield J. Am. Chem. Soc., 85:2149-2146 (1963)). Equipment for automated synthesis of polypeptides is commercially available from suppliers such as PE Corp. (Foster City, Calif.), and may generally be operated according to the manufacturer's instructions. The synthesized peptides can then be cleaved from the resin, and purified, e.g., by preparative high performance liquid chromatography (see Creighton, Proteins Structures and Molecular Principles, 50-60 (1983)). The composition of the synthetic polypeptides or of subfragments of the polypeptide, may be confirmed by amino acid analysis or sequencing (e.g., the Edman degradation procedure; see Creighton, Proteins, Structures and Molecular Principles, pp. 34-49 (1983)).

In addition, nonclassical amino acids or chemical amino acid analogs can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, omithine, norleucine, norvaline, hydroxy-proline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

The binding ligand or reporter peptide may also be joined to their respective coils via a linking group. The linking group can be a chemical crosslinking agent, including, for example, succinimidyl-(N-maleimidomethyl)-cyclohexane-1-carboxylate (SMCC). The linking group can also be an additional amino acid sequence(s), including, for example, a polyalanine, polyglycine or similarly, linking group.

Other chemical linkers include carbohydrate linkers, lipid linkers, fatty acid linkers, polyether linkers, e.g., PEG, etc. For example, poly(ethylene glycol) linkers are available from Shearwater Polymers, Inc. Huntsville, Ala. These linkers optionally have amide linkages, sulfhydryl linkages, or heterofunctional linkages.

Possible chemical modifications of the binding ligand or the reporter peptide and their respective coils also include derivitization with polyethylene glycol (PEG) to extend time of residence in the circulatory system and reduce immunogenicity, according to well known methods (See for example, Lisi, et al., Applied Biochem. 4:19 (1982); Beauchamp, et al., Anal. Biochem. 131:25 (1982); and Goodson, et al., Bio/Technology 8:343 (1990)).

A domain, for example a non-peptide reporter label such as a fluorescent dye, is typically linked to a coil by chemical conjugation. Such domains are conjugated to a member of a coil-coil binding pair using methods known in the art ( see, e.g., Bioconjugate Techniques, Hermanson, Ed., Academic Press (1996)).

C. Assembly of Bifunctional or Multifunctional Polypeptides

Once each domain comprising different functions, e.g., a binding ligand and a reporter molecule, has been linked to their respective coil, the components are assembled to form a bifunctional or multifunctional polypeptide. Preferably the linked coils are incubated in conditions in which they self-assemble by association of their respective coils, for example, incubation at room temperature in Dulbecco's Phosphate Buffered Saline (PBS), for 15 minutes.

In some embodiments, a polypeptide that is linked to one of the coils may also be capable of undergoing spontaneous multimeric assembly. For example, such a moiety may form a dimer or multimer with itself, or a with a different polypeptide. This property can further enhance the sensitivity of the bifunctional or multifunctional polypeptide. An example of a self-multimerizing polypeptide is a ferritin polypeptide or a viral coat protein derived from viruses such as poliovirus, Hepatitis B, Cow pea mosaic virus, Johnsong Grass Mosaic Virus coat protein, polyoma viruses of many species, and a grouper β nodaviruses of different species capsid protein. These form multimers with tens of subunits. Alkaline phosphatase, which is also an enzyme, is an example of a protein which spontaneously dimerizes. As appreciated by one of skill in the art, additional self-multimerizing proteins are known and can be identified. For example, such a protein can be identified by determining the ability of a polypeptide to form a multimeric complex after incubation at room temperature. The length of such an incubation is typically 15 to 30 minutes, although it may not require that length of time for an assembled structure to form.

In some embodiments, a multifunctional polypeptide comprises subunits in which individual members of the coil binding pair are linked to different proteins. For instance, one of the coil pair members, e.g., an E-coil, can be individually linked to a binding ligand such as an scFv, a fluorobody, a chromobody, or to a reporter molecule such as a fluorescent or colored protein. The other member of the coil can be individually linked to one or more polypeptides that undergo self-assembly to form a multimer, e.g., soluble ferritin, or two polypeptides that form dimers. The E-coil-binding ligand and E-coil reporter molecule are mixed with the K-coil-linked, self-multimerizing polypeptide (or polypeptides). A complex is thereby formed that comprises the multimeric, self-assembled complex linked via the coil-coil interactions to both the reporter molecule and the binding ligand. The proportions of reporter and binding ligand in the end complex can be modulated by controlling the ratio of reporter to binding ligand included in the assembling complex.

Similarly, in other embodiments, multiple binding ligands may be included in the multifunctional polypeptide. For example, one or more binding ligands may be linked individually to a member of a coil-coiled binding pair. The multiple ligands can then be joined to a reporter molecule and/or a multimerizing domain linked to the second member of the coil-coiled binding pair via the coil-coil interaction. For example, multiple fluorobodies may be linked to a mutimerization domain via a coil-coil interaction. In this case, the binding ligand and the reporter molecule are the same, e.g., the fluorobody. Similarly, in chromobodies the reporter (color) and the binding ligand are embodied in the same molecule.

D. Detection of Protein of Interest

In some embodiments, the bifunctional or multifunctional polypeptides of the invention can be used to screen for the presence of a particular antigen in a sample. The polypeptides can be used, for example, in western blot assays, ELISAs and other method to detect the presence of a target molecule or antigen. Methods of detecting antigen are well known in the art and are described in, e.g., Harlow and Lane, ANTIBODIES, A LABORATORY MANUAL, Cold Spring Harbor Publication, New York (1999). For example, a sample comprising the antigen may be incubated with an assembled bifunctional or multifunctional polypeptide comprising a binding ligand (e.g., an antibody) and a reporter molecule (e.g., GFP), and binding of the antibody to the antigen detected by detecting activity of the reporter polypeptide. Alternately, the sample comprising the antigen may first be incubated with a binding ligand (e.g., an antibody) linked to a coil (e.g., an E coil). The antigen-binding ligand complex is subsequently incubated with a reporter polypeptide linked to a coil (e.g., a K coil). Binding of the antibody to the antigen is detected by detecting the activity of the reporter polypeptide (i.e., label). Fluorobodies, which comprise both reporter function and binding function, may also be used, and considerable signal amplification can be obtained by the multimerization of the reporter and binding functions.

Means of detecting labels are well known to those of skill in the art. Thus, for example, where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple calorimetric labels may be detected simply by observing the color associated with the label. Thus, in various dipstick assays, conjugated gold often appears pink, while various conjugated beads appear the color of the bead.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

The bifunctional or multifunctional polypeptides can offer increased sensitivity relative to other immunoassay reagents. For example, bifunctional polypeptide comprising multimerized polypeptides can be used to detect low levels of target molecule. In such an application, the bifunctional polypeptide bound to target molecule can be detected using a reagent, typically an antibody, that binds specifically to a polypeptide that has undergone multimerization. The presence of multiple copies of the polypeptide thus amplifies the signal. Accordingly, the bifunctional polypeptide has increased sensitivity relative to detection reagents comprising only a single copy of the polypeptide.

A multifunctional polypeptide comprising a binding ligand, a reporter molecule, and a spontaneously assembling domain also offers increased sensitivity.

E. Kits

The invention also provides kits that includes the bifunctional or multifunctional polypeptides of the invention or individual components. The kit can comprises the assembled polypeptide, or alternatively can comprises individual components. For example, the kit could comprise a binding ligand linked to one of the coil domain. The second polypeptide that is linked to the second coil domain can be included as an individual component of the same kit, or alternatively, may be provided in a different kit. The kit can also includes instructions for using the polypeptides and accessory reagents such as detection reagents.

F. Pharmaceutical Compositions

In other embodiments, the bifunctional or multifunctional polypeptide can be formulated in a pharmaceutically acceptable solution for administration to a cell or an animal, either along, for diagnostic or therapeutic purposes. The reagents can be administered alone or in combination with other agents. Further, the polypeptide can be administered in an assembled form as an individual components. When a bifunctional or multifunctional polypeptide is administered therapeutically, it typically comprises a binding ligand, i.e., a targeting moiety, and a therapeutic moiety, e.g., a cytoxic moiety, a growth factor, a cytokine, or a drug, which can be joined via coiled-coil binding.

It will also be understood that, if desired, the bifunctional polypeptides of the present invention may be administered in combination with other agents as well, such as, e.g., other proteins or polypeptides or various pharmaceutically-active agents. Any other components may be included, provided that the additional agents do not cause a significant adverse effect upon contact with the target cells or host tissues.

Formulation of pharmaceutically-acceptable excipients and carrier solutions is well-known to those of skill in the art, as is the development of suitable dosing and treatment regiments for using the particular compositions described herein in a variety of treatment regimens, including e.g., parenteral, intravenous, inhalation, intramuscular, and rectal administration and formulation. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, intraperitoneally, intravesically or intrathecally. The formulations can be presented in unit-dose or multi-dose sealed containers, such as ampules and vials. Injection solutions and suspensions can be prepared from sterile powders, granules, and tablets.

The dose administered to a patient, in the context of the present invention should be sufficient to effect a beneficial therapeutic response in the patient over time. The dose will be determined by the efficacy of the particular bifunctional or multifunctional polypeptide employed and the condition of the patient, as well as the body weight or surface area of the patient to be treated. The size of the dose also will be determined by the existence, nature, and extent of any adverse side-effects that accompany the administration of the polypeptide in a particular patient.

In determining the effective amount of the polypeptide to be administered, the physician can evaluate circulating plasma levels of the polypeptide, toxicities of the polypeptide, progression of the disease, and the production of antibodies against the polypeptide.

For administration, polypeptides of the present invention can be administered at a rate determined by the LD-50 of the polypeptide, and the side-effects of the polypeptide at various concentrations, as applied to the mass and overall health of the patient. Administration can be accomplished via single or divided doses. Administration can be accomplished parenterally, intravenously, intramuscularly, or even intraperitoneally as described in U.S. Pat. Nos. 5,543,158; 5,641,515 and 5,399,363, intranasal sprays, inhalation, and/or other aerosol delivery vehicles as described in U.S. Pat. Nos. 5,756,353, 5,804,212, Takenaga et al., 1998, U.S. Pat. Nos. 5,725,871, 5,780,045.

### EXAMPLES

These examples demonstrate the preparation and use of bifunctional and multifunctional polypeptide.

Exemplary Materials and Methods

The genes encoding anti lysozyme single-chain antibodies D1.3 and HyHEL10 are described in Neri, et al., J. Mol. Biol. 246:367-373, 1995. These two single-chain antibodies can bind simultaneously to lysozyme. Using methods known in the art, oligonucleotides corresponding to the amino acid sequence of the scFvs were used to create a recombinant construct encoding a E coil fused to the C terminus of the scFv in the pDAN5 vector. Recombinant fusion proteins were expressed and purified using Ni—NTA beads (Qiagen) according to methods known in the art. Reagents were used at 1 g/200 μL (˜150 nM), lysozyme at 500 ng/well corresponds to 150 nM solution. FRET was calculated as an acceptor signal (excitation at 360 nm and emission at 535 nm) over donor signal (excitation at 485 nm and emission at 535 nm).

The anti ubiquitin single-chain antibody aU4 used was selected from a phagemid library (Sheets, et al., Proc. Natl. Acad. Sci. USA 95:6157-6162, 1998) against purified bovine ubiquitin (Sigma). Its binding epitope maps to the last 15 amino acids of ubiquitin, but does not include the C-terminal carboxyl group.

Using methods known in the art, oligonucleotides corresponding to the amino acid sequence of the coils were used to create a recombinant construct encoding a K coil fused to the C terminus of the scFv and a recombinant construct encoding a E coil fused to the N terminus of GFP. The K coil-scFv polypeptide and E coil-GFP polypeptide were expressed using methods known in the art. The two polypeptides were allowed to self assemble using standard reaction conditions, Dulbecco's Phosphate Buffered Saline (PBS), room temperature, 15 minutes.

### Example 1

### Preparation and Use of Bifunctional Polypeptides

Labeling of scfsv with Fluorescent Organic Dyes Using Coiled-Coil Interaction

Anti-lysozyme single-chain antibody HyHEL10-Ecoil fusion protein and synthetic K-coil labeled with Alexa488 fluorescent dye (Molecular Probes, Eugene, Oreg.) were used in this experiment. Incubation of indicated mixtures was performed at room temperature for 15 minutes in PBS buffer. The incubated mixtured were then electrophoresed on a native gel. The gel was scanned on a fluoroscanner to visualize Alexa488 fluorescent dye.

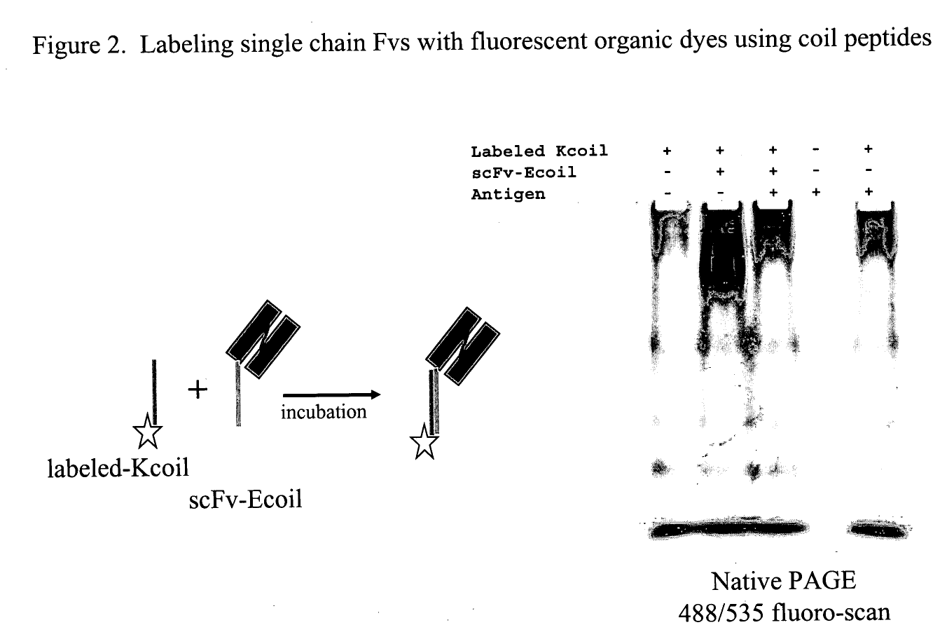

The results of this experiment (FIG. 2) show that the coil fusions spontaneously associated via a coil-coil interaction following incubation, as the scFv became labeled with the fluorescent dye.

One Step Western Blot

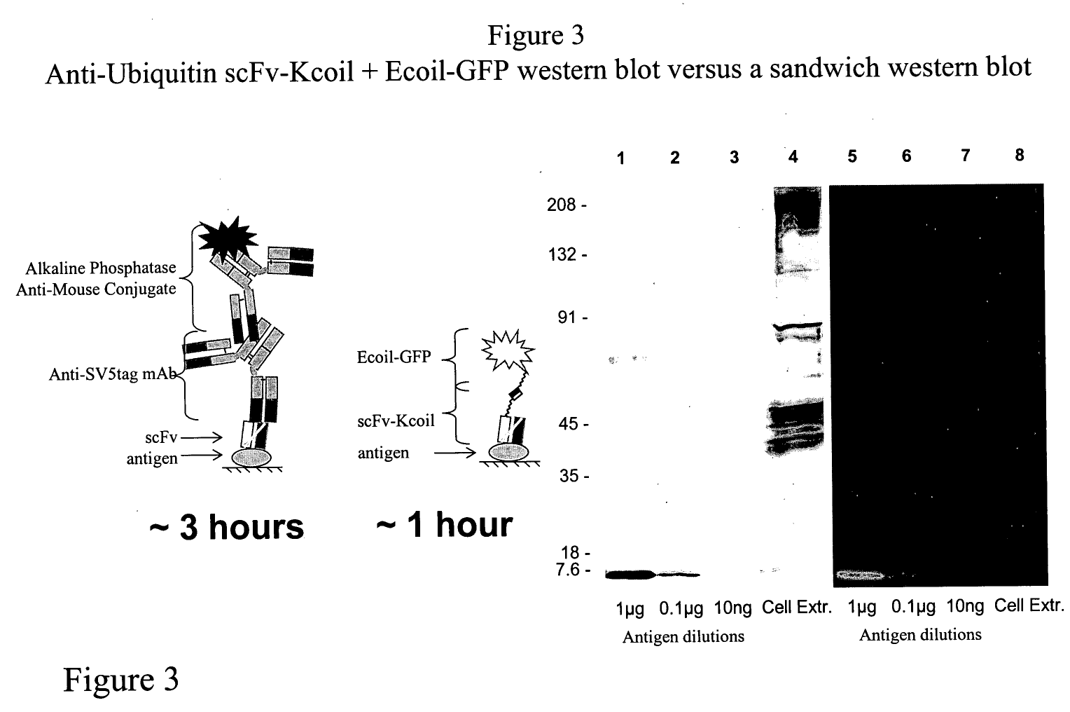

This example shows detection of an antigen bound to a solid support using a bifunctional polypeptide of the invention comprising a reporter domain and a binding ligand domain, each of which is fused to a coil. An anti-ubiquitin single-chain antibody fused to a K coil (aU4-Kcoil fusion protein) and Ecoil-GFP protein were used in a western blot (FIG. 3). Three dilutions of purified ubiquitin and HeLa cell extract (Cell Extr.) were electrophoresed on SDS-PAGE and blotted onto nitrocellulose membrane. Lanes 1-4 were incubated with single chain antibody aU4 followed by incubations with anti-HSVtag monoclonal antibody and anti-mouse-AP conjugate and the signal was developed with NBT/BCIP substrate. Lanes 5-8 were incubated with aU4-Kcoil fusion protein followed by incubation with Ecoil-GFP protein and the membrane was photographed under a 485 nm lamp. Molecular weight markers in kDa are indicated on the left. Multiple bands in the cell extract from HeLa cells above 35 kDa are likely to be ubiquitinated proteins. FIG. 3 shows that the coil on the scFv interacted with the coil on the GFP, which resulted in labeling of the scFv with GFP.

One Step Detection of Antigen in Solution Using Fluorescent Resonance Energy Transfer (FRET) Between Green Fluorescence Protein (GFP) and Blue Fluorescence Protein (BFP)

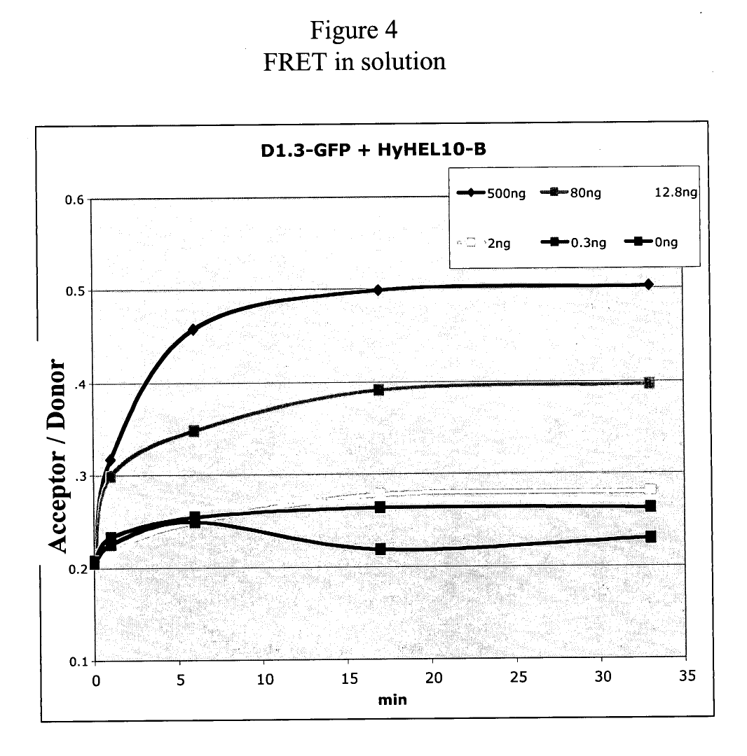

This example shows the detection of an antigen in solution using a bifunctional polypeptide of the invention. Anti-lysozyme single-chain antibodies D1.3-Kcoil and HyHEL10-Kcoil fusion proteins were first labeled by 15 minute incubation with Ecoil-GFP and Ecoil-BFP fusion proteins, respectively. They were then mixed together in a 1:1 molar ratio. Various indicated amounts of purified lysozyme were then added and FRET signal was measured over indicated period of time. The results of this experiment are shown in FIG. 4 and show that only the correct scFv-coil fluorescent protein fusions provided FRET upon antigen binding.

### Example 2

### Synthesis and Characterization of Multifunctional Polypeptides

One Step ELISA

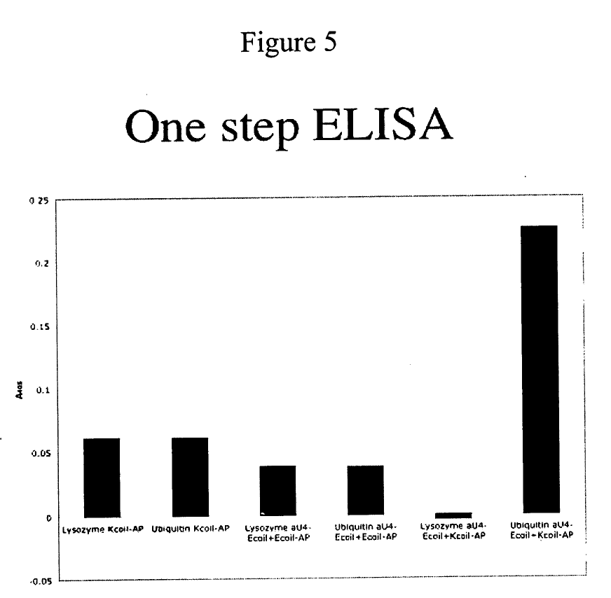

Anti-ubiquitin single-chain antibody aU4-Ecoil, Ecoil-Alkaline Phosphatase and Kcoil-Alkaline Phosphatase fusion proteins were used. The Ecoil-Alkaline Phosphatase is used as a negative control as it is unable to bind to the aU4-Ecoil. Microtiter plate wells were coated either with ubiquitin (specific target) or lysozyme (non-specific), blocked with 4% fish gelatin and washed. Indicated fusion proteins were incubated in 1:1 molar ratio for 15 minutes in PBS at room temperature, added to the wells and allowed to bind for 1 hour. Wells were then washed, alkaline phosphatase substrate (PNPP) was added and signal was detected at 405 nm. The results are shown in FIG. 5. The experiment indicates that only in the case where the alkaline phosphatase and the scFv were joined by virtue of the coils does the ELISA show specific signals. In this experiment, the alkaline phosphatase also provides the additional function of dimerization in addition to the addition of an enzymatic activity.

Synthesis and Characterization of a Fluoroferritin Using Coiled Coils for Detection Purpose

E-coil tagged GFP was expressed in BL21(DE3) using a pET vector, and the crude protein concentrated to ca. 22 mg/ml. N-terminally E-coil tagged ferritin and N-terminally GFP tagged ferritin were each expressed and the crude proteins concentrated to ca. 22 mg/ml. Target protein concentrations were estimated by SDS-PAGE densitometry. Fluoroferritin (ferritin containing fluorescent proteins) containing approximately 3 E-coil-ferritin moieties and 21 GFP-ferritin moieties per 24-mer holoferritin assembly were prepared by mixing ferritin fusion proteins in a 21:4 ratio (125 ul of 6.7 uM E-coil ferritin and 1800 ul of 46 uM GFP ferritin), denatured in with 17 ml of 9 M Urea, (final concentration of urea 8.1 M) and refolded by 10-fold dilution in 150 mM TRIS buffer pH 7.5, 150 mM NaCl, 10% glycerol (TNG Buffer). E-coil GFP staining solution (ca. 8.5×10−11 M in E-coil GFP) was prepared by diluting 24 ul of lysate containing 0.21 mg/ml crude E-coil GFP in 20 ml TNG buffer. Fluoroferritin staining solution 3.2×10⁻¹¹ M in fluoroferritin, (24-mer assemblies ca. 9.8×10⁻¹¹ M in E-coil ferritin subunits and 6.8×10⁻¹⁰ M in GFP-ferritin subunits), was prepared by mixing 320 μl of the re-natured fluoroferritin and 20 ml of TNG. One microliter volumes of eight serial 2-fold dilutions of BFP-K-coil with concentrations ranging from 2.0×10⁻¹ mg/ml down to 1.6×10⁻³ mg/ml were transferred to two nitrocellulose membranes, which were subsequently blocked with one 20 ml volume of 1% BSA 1 h, washed 1 h with three 20 ml volumes of TRIS buffer, stained 1 h with E-GFP staining solution or fluoroferritin staining solution, and imaged by fluorescence. In this experiment, the fluorescence of the BFP is not used. The concentration of E-coil moities was about equal in both staining solutions, but the fluoroferritin enabled more facile detection of the BFP-K-coil, i.e., the signal was amplified, relative to staining by E-coil GFP, presumably because of (1) the increased avidity due to multiple E-coil binding domains per holoferritin, and (2) increased labeling ratio of the holoferritin (ca. 7 GFP moieties per E-coil moieties). In contrast, each E-coil GFP has only one fluorescence unit per binding domain.

This example thus shows: 1) the E-coil attached to the ferritin is functional and can bind to its partner K-coil when fused to GFP. Given that previous experiments have shown that coils can be used to link scFvs to other scFvs, GFP or to alkaline phosphatase, the coil on the ferritin could also be used to bind to a scFv, a fluorobody, or a chromobody with a K-coil, which could in turn provide the binding specificity of the fluoroferritin; 2) GFP multimerized by the ferritin provides a much stronger signal than single GFP alone, even when the molarity of the coils is identical.

Synthesis and Characterization of a Multivalent scFv-Ferritin

Using methods known in the art, oligonucleotides corresponding to the amino acid sequence of the coils are used to create a recombinant construct encoding a K coil fused to the N-terminus of a soluble L-subunit bullfrog red cell ferritin and a recombinant construct encoding a E coil fused to the C terminus of a scFv. The K coil-ferritin polypeptide and E coil-scFv polypeptide are each separately expressed using methods known in the art. Ferritin spontaneously assembles into a 24-subunit spherical multimeric protein. Consequently, the assembled ferritin multimer displays 24 K coil peptides. The E coil-scFv polypeptide can be mixed with the K coil-ferritin and allowed to self assemble using standard reaction conditions.

Synthesis and Characterization of a Multivalent scFv-Ferritin-GFP

Using methods known in the art, oligonucleotides corresponding to the amino acid sequence of the coils are used to create a recombinant construct encoding a K coil fused to the N-terminus of a soluble L-subunit bullfrog red cell ferritin, a recombinant construct encoding an E coil fused to the C terminus of a scFv, and a recombinant construct encoding an E coil fused to the C-terminus of GFP. The K coil-ferritin polypeptide, the E coil-scFv polypeptide, and the GFP-E coil polypeptide are each separately expressed using methods known in the art. Ferritin spontaneously assembles into a 24-subunit spherical multimeric protein. Consequently, the assembled ferritin multimer displays 24 K coil peptides. The E coil-scFv polypeptide and the GFP-E coil polypeptide can be mixed with the K coil-ferritin and allowed to self assemble using standard reaction conditions, creating a ferritin which has displayed on its surface both scFv and GFP, the ratio between them being dependent upon the amounts added to the mixture.

Synthesis and Characterization of a Multivalent scFv-Ferritin-GFP

Using methods known in the art, and described above, oligonucleotides corresponding to the amino acid sequence of the coils are used to create a recombinant construct encoding a K coil fused to the N-terminus of a soluble L-subunit bullfrog red cell ferritin, a recombinant construct encoding an E coil fused to the C terminus of a scFv, and a recombinant construct encoding N-terminally GFP tagged ferritin. The K coil-ferritin polypeptide, the E coil-scFv polypeptide, and the GFP-ferritin polypeptide are each separately expressed using methods known in the art. Ferritin spontaneously assembles into a 24-subunit spherical multimeric protein. Consequently, by providing different proportions of GFP-ferritin and K-coil-ferritin, the assembled ferritin multimer variably displays K coils or GFP molecules, so varying signal intensity and avidity. The E coil-scFv polypeptide can be mixed with the K coil-ferritin and allowed to self assemble using standard reaction conditions, creating a ferritin which had displayed on its surface both scFv and GFP, the ratio between them being dependent upon the amounts added to the mixture.

Synthesis and Characterization of a Multivalent Fluorobody-Ferritin

Using methods known in the art, and described above, oligonucleotides corresponding to the amino acid sequence of the coils are used to create a recombinant construct encoding a K coil fused to the N-terminus of a soluble L-subunit bullfrog red cell ferritin and a recombinant construct encoding an E coil fused to the C terminus of a fluorobody. The K coil-ferritin polypeptide and the E coil-fluorobody are each separately expressed using methods known in the art. Ferritin spontaneously assembles into a 24-subunit spherical multimeric protein. The K coil-ferritin can be mixed with the E-fluorobody under standard conditions to create a multimeric fluorobody.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference in their entirety for all purposes.

1. A bifunctional polypeptide comprising a binding ligand linked to a first member of a coil-coil binding pair and a reporter molecule linked to the second member of a coil-coil binding pair, wherein binding between the first coil domain and the second coil domain joins the binding ligand to the reporter molecule.

2. The bifunctional polypeptide of claim 1, wherein the coil-coil binding pair is an E coil and a K coil.

3. The bifunctional polypeptide of claim 1, wherein the coil-coil binding pair is an A coil and a B coil.

4. The bifunctional polypeptide of claim 1, wherein the first and the second members of the coil-coil binding pair are each at least 35 amino acids in length.

5. The bifunctional polypeptide of claim 1, wherein the binding ligand is an antibody selected from the group consisting of an scFv, an Fab fragment, an isolated V_{H}, and an isolate V_{L}.

6. The bifunctional polypeptide of claim 1, wherein the binding ligand is a peptide.

7. The bifunctional polypeptide of claim 1, wherein the binding ligand is a receptor.

8. The bifunctional polypeptide of claim 1, wherein the reporter molecule is a fluorescent protein or chromophoric protein.

9. The bifunctional polypeptide of claim 8, wherein the fluorescent protein is green fluorescent protein.

10. The bifunctional polypeptide of claim 8, wherein the fluorescent protein is red fluorescent protein.

11. The bifunctional polypeptide of claim 1, wherein the reporter molecule is a fluorescent dye.

12. The bifunctional polypeptide of claim 1, wherein the reporter molecule is an enzyme.

13. The bifunctional polypeptide of claim 12, wherein the enzyme is horseradish peroxidase.

14. The bifunctional polypeptide of claim 12, wherein the enzyme is alkaline phosphatase.

15. The bifunctional polypeptide of claim 1, wherein the reporter molecule is a biotin binding protein.

16. The bifunctional polypeptide of claim 1, wherein the reporter molecule has luminescent activity.

17. The bifunctional polypeptide of claim 16, wherein the reporter molecule is luciferase.

18. A multifunctional polypeptide comprising:
a first member of a coil-coil binding pair linked to a binding ligand; and
a second member of the coil-coil binding pair linked to a polypeptide that undergoes spontaneous multimerization to form a self-assembled complex;
wherein binding between the first member of the coil-coil binding pair and the second member of the coil-coil pair joins the binding ligand and the self-assembled complex.

19. The multifunctional polypeptide of claim 18, further comprising a reporter molecule that is individually linked to a first member of the coil-coil binding pair, wherein the coil-coil binding interaction joins the reporter molecule to the multifunctional polypeptide.

20. The multifunctional polypeptide of claim 18, further comprising a second binding ligand that is individually linked to a first member of a coil-coil binding pair, wherein the second binding ligand binds to an epitope different from the first binding ligand, and wherein the coil-coil binding interaction joins the self-assembled complex to the first and second binding ligands.

21. The multifunctional polypeptide of claim 18, further comprising a second polypeptide individually linked to a second member of the coil-coil binding pair, wherein the second polypeptide undergoes spontaneous multimerization with the first polypeptide to form the self-assembled complex, and wherein the coil-coil binding interaction joins the self-assembled complex to the binding ligand.

22. The multifunctional polypeptide of claim 18, wherein the polypeptide is a soluble ferritin subunit.

23. The multifunctional polypeptide of claim 18, wherein the polypeptide is a viral coat protein.

24. The multifunctional polypeptide of claim 18, wherein the coil-coil binding pair is an E coil and a K coil.

25. The multifunctional polypeptide of claim 18, wherein the coil-coil binding pair is an A coil and a B coil.

26. The multifunctional polypeptide of claim 18, wherein the first and the second members of the coil-coil binding pair are each at least 35 amino acids in length.

27. The multifunctional polypeptide of claim 18, wherein the binding ligand is an antibody selected from the group consisting of a single chain Fv, an Fab, an isolated V_{H}, and an isolated V_{L}.

28. The multifunctional polypeptide of claim 18, wherein the binding ligand is a peptide.

29. The multifunctional polypeptide of claim 18, wherein the binding ligand is a fluorobody.

30. The multifunctional polypeptide of claim 18, wherein the binding ligand is a receptor.

31. The multifunctional polypeptide of claim 18, wherein the reporter molecule is a fluorescent protein.

32. The multifunctional polypeptide of claim 18, wherein the fluorescent protein is green fluorescent protein.

33. The multifunctional polypeptide of claim 18, wherein the fluorescent protein is red fluorescent protein.

34. The multifunctional polypeptide of claim 18, wherein the reporter molecule is an enzyme.

35. The multifunctional polypeptide of claim 18, wherein the reporter molecule is a biotin binding protein.

36. The multifunctional polypeptide of claim 18, wherein the reporter molecule is luciferase.

37. A kit comprising:
a binding ligand linked to a first member of a coil-coil binding pair; and
a reporter molecule linked to a second member of a coil-coil binding pair.

38. The kit of claim 37, wherein the binding ligand is an antibody.

39. The kit of claim 37, wherein the binding ligand is a peptide.

40. The kit of claim 37, wherein the binding ligand is a fluorobody.

41. The kit of claim 37, wherein the reporter molecule is a fluorescent protein.

42. The kit of claim 37, wherein the reporter molecule is an enzyme.

43. The kit of claim 37, wherein the reporter molecule is a biotin binding protein.

44. The kit of claim 37, wherein the reporter molecule is luciferase.

45. A kit comprising:
a first subunit that is a first member of a coil-coil binding pair linked to a binding ligand; and
a second subunit that is a second member of the coil-coil binding pair linked to a polypeptide that undergoes spontaneous multimerization.

46. The kit of claim 45, further comprising a third subunit that is a first member of the coil-coil binding pair linked to a reporter molecule.

47. The kit of claim 45, wherein the polypeptide that undergoes spontaneous multimerization is soluble ferritin.

48. The kit of claim 45, wherein the polypeptide that undergoes spontaneous multimerization is a viral coat protein.

49. The kit of claim 45, wherein the binding ligand is an antibody selected from the group consisting of an scFV, an Fab, a V_{H} region and a V_{L} region.

50. The kit of claim 45, wherein the binding ligand is a fluorobody.

51. The kit of claim 45, wherein the binding ligand is a peptide.

52. The kit of claim 45, wherein the binding ligand is a receptor.

53. The kit of claim 45, wherein the reporter molecule is a polypeptide selected from the group consisting of a fluorescent protein and an enzyme.

54. The kit of claim 53, wherein the reporter molecule is a fluorescent protein or an enzyme.

55. A method of making a multifunctional polypeptide, the method comprising:
providing a binding ligand linked to a first member of a coil-coil binding pair;
providing a molecule linked to a second member of a coil-coil binding pair, wherein the molecule is a reporter molecule or a spontaneously multimerizing polypeptide; and
incubating the binding pair under conditions in which the first binding pair member specifically binds to the second binding pair member, thereby assembling the bifunctional polypeptide.

56. A method of screening for the presence of an antigen, the method comprising:
incubating a sample comprising the antigen with a bifunctional polypeptide comprising a binding ligand linked to a first member of a coil-coil binding pair and a reporter molecule linked to the second member of the coil-coil binding pair, wherein the binding ligand is joined to the reporter polypeptide by the binding interaction between the binding pair members;
wherein the antigen and the bifunctional polypeptide are incubated under conditions in which the antigen specifically binds to the binding ligand; and
detecting activity of the reporter molecule, thereby detecting the presence of the antigen.

57. A method of screening for the presence of an antigen, the method comprising:
incubating a sample comprising the antigen with a binding ligand linked to a first member of a coil-coil binding pair under conditions in which the antigen specifically binds to the binding ligand and subsequently incubating the sample with a reporter molecule linked to the second member of the coil-coil binding pair, wherein the binding ligand becomes joined to the reporter molecule by the binding interaction between the binding pair members; and
detecting activity of the reporter polypeptide, thereby detecting the presence of the antigen.

58. A method of screening for the presence of an antigen, the method comprising:
(a) incubating a sample comprising the antigen with a bifunctional polypeptide comprising:
(i) a binding ligand linked to a first member of a coil-coil binding pair; and
(ii) a polypeptide that undergoes spontaneous multimerization linked to the second member of a coil-coil binding pair, wherein binding between the first coil domain and the second coil domain joins the binding ligand and the spontaneously multimerizing polypeptide,
wherein the antigen and the bifunctional polypeptide are incubated under conditions in which the antigen specifically binds to the binding ligand; and
(b) detecting the presence of the bifunctional polypeptide, thereby detecting the presence of the antigen.

59. A method of screening for the presence of an antigen, the method comprising:
incubating a sample comprising the antigen with a binding ligand linked to a first member of a coil-coil binding pair under conditions in which the antigen specifically binds to the binding ligand and subsequently incubating the sample with a second polypeptide linked to a second member of the coil-coil binding pair, wherein the second polypeptide undergoes spontaneous multimerization;
wherein the binding ligand is joined to the second polypeptide by the binding interaction between the coil-coil binding pair members to form a multifunctional polypeptide; and
detecting the presence of the multifunctional polypeptide, thereby detecting the presence of the antigen.
